(19) United States
(12) Patent Application Publication
(10) Pub. No.: US 2011/0301452 A1
(43) Pub. Date: Dec. 8, 2011

(54) Microcapsule for local treatment of a tumor and method for positioning a magnetic gradient field guiding magnetic nanoparticles to a target location as well as apparatus for positioning a magnetic gradient field

(76) Inventors: Michael Maschke, Lonnerstadt (DE); Klaus Klingenbeck, Aufsess (DE)

(21) Appl. No.: 13/151,330

(22) Filed: Jun. 2, 2011

(30) Foreign Application Priority Data
Jun. 7, 2010 (DE) 10 2010 022 926.1

(51) Int. Cl. A61B 5/055; A61K 31/704; A61P 35/00; A61N 2/00; A61K 9/50; A61K 31/136; B82Y 5/00

(52) U.S. Cl. 600/424; 424/452; 424/1.11; 514/34; 514/649; 600/9; 977/906

(57) ABSTRACT

A microcapsule for the local treatment of a tumor is proposed. The microcapsule has a support material forming a casing for the microcapsule, an active agent that damages tumor cells, a marker material suitable for use as an x-ray marker, and at least one magnetic nanoparticle. The active agent in particular destroys the tumor cells.

### CROSS REFERENCE TO RELATED APPLICATIONS

This application claims priority of German application No. 10 2010 022 926.1 filed Jun. 7, 2010, which is incorporated by reference herein in its entirety.

### FIELD OF THE INVENTION

The invention relates to a microcapsule for the local treatment of a tumor, a method for positioning a magnetic gradient field guiding magnetic nanoparticles, in particular magnetic nanoparticles of a microcapsule, to a target location and/or holding them at the target location as well as an associated apparatus.

### BACKGROUND OF THE INVENTION

Cancers have always represented a challenge for modern medicine. Conventional cancer therapies can generally provide the following measures:

1. surgical removal of the tumor,
2. chemotherapy,
3. radiation therapy and
4. a combination of the three measures listed above.

Despite all the advances achieved with the abovementioned measures many patients may experience recurring tumors or metastases. The forms of therapy described above may be repeated depending on the state of health of the patient, their age and the type of tumors. However it may be that a patient is “therapied out”, which means to say that the abovementioned conventional therapeutic measures can no longer be used, as the patient can no longer tolerate the burden of such therapies physically and/or mentally.

For such “therapied out” patients it is known to administer powerful painkillers, some of which can also be introduced directly into the region of the tumor by way of a catheter. However this only serves to relieve the pain not to provide a cure.

Therefore what is known as tumor ablation has recently been proposed as a new treatment measure. Provision is made here for guiding a tool, for example a catheter or biopsy needle, to the tumor or metastasis and to attack the tumor with the aid of different forms of energy or by injecting alcohol.

What is known as thermoablation is described here by way of example. For the purposes of thermoablation it is possible to use radio frequency (RF), microwave, ultrasound and/or laser energy. In this process the tumor cells are destroyed by high temperatures, while healthy tissue is preserved. In the case of radio frequency ablation (RFA) an interventional radiologist introduces a thin needle into the tumor of a patient with the aid of imaging technologies. Radio frequency energy is transmitted from the tip of the needle to the target tissue, where it generates massive heat, thereby destroying the tumor. The destroyed tissue shrinks and slowly forms a scar.

Depending on the size of the tumor an RFA can shrink it or destroy it, thereby extending the life of a patient and improving their quality of life considerably. As RFA is a local method, which has little or no detrimental effect on the healthy tissue, the treatment can be repeated a number of times.

The pain caused by tumors and other symptoms of weakness are alleviated by reducing the size of the tumor or treating newly occurring tumors. Even though the tumors themselves often cause no pain, they can press on nerves or major organs, in some instances causing enormous pain. RFA can be used for small to medium tumors, see also the article by Zhengium Liu, “Radiofrequency Tumor Ablation”, AJR: 184, April 2005, pp. 1347 to 1352.

One major disadvantage of the above ablation therapy is that it can only be used for relatively small tumors. However a very large proportion of tumors are only discovered at a relatively late stage and can therefore no longer be treated using this form of therapy.

One alternative to ablation is what is known as radioembolization, also referred to as selective internal radiotherapy (SIRT). Here the vessels are blocked off using radioactive microcapsules, which are frequently spherical in shape. These spheres have the approximate diameter of five red blood corpuscles and lodge in the blood vessels of the tumor, from whence they emit their radiation, which damages and in particular destroys the tumor cells.

The radioactive isotope yttrium-90 is frequently used, with the microcapsules containing the radioactive isotope being produced and brought to the treatment clinic immediately before the intervention. Yttrium-90 is preferred, as it is a pure beta emitter. This means that the unwanted radiation exposure for people round about is low and 90% of the particle radiation energy is deposited in the tissue within a radius of approx. 9 to 11 mm. The relatively short physical half-life means that only a relatively short inpatient stay is required in a clinic specializing in nuclear medicine, for example for around 48 hours.

The production of such radioactive therapeutic active agents or corresponding microcapsules is described for example in US 2004/00776582 A1.

Radioembolization is performed as an image-controlled therapy by an interventional radiologist working with a practitioner of nuclear medicine. A local anesthetic is applied after which a thin catheter is introduced into the artery through a small puncture in the skin in the groin. The movement of the catheter to the target location, in other words the tumor, is then monitored by means of fluoroscopy. When a liver tumor is being treated, the advance of the catheter into the liver artery (Arteria Hepatica) for example can be monitored by fluoroscopy. The radioactive isotope is injected through the catheter in the form of microcapsules, in particular microspheres, directly into the branches of the artery that supply the tumor, in particular the liver tumor. These microspheres lodge in the tumor vessels, from whence they emit their radiation, which destroys the tumor cells. Since the radiation is restricted to this region, it can also be applied in higher doses, without also adversely affecting healthy tissue.

Radioembolization is generally a palliative treatment method, which means it does not cure cancer. However patients benefit greatly from this procedure, as it increases their life expectancy and improves their quality of life. It is a relatively new therapeutic approach but has already demonstrated success in the treatment of primary tumors or metastases. Fewer side effects have been observed up to now with this treatment method than for example with conventional chemotherapies.

Radioembolization is employed for example for hepatocellular carcinomas (HCC), cholangiocellular carcinomas and liver metastases, for example with bowel and breast cancer or other malignant tumors. The most frequent side effect is tiredness, which can last for seven to ten days.

One disadvantage of this procedure is that if the radioactive microcapsules enter the lung, gall bladder or stomach, they can cause radiation damage there.

A further new, very promising, treatment method is what is known as “magnetic drug targeting”, where magnetic nanoparticles loaded with chemotherapy drugs are introduced into the tumor by way of a catheter or puncture needle. The magnetic nanoparticles are then concentrated by means of a magnet in the region with the strongest field gradient and then release the chemotherapy drug in the tumor. Magnetic nanoparticles for therapy purposes are known for example from U.S. Pat. No. 6,514,481 A1.

One major disadvantage of the solution to date is that the magnetic nanoparticles, which are frequently iron-oxide-based are only clearly visible in magnetic resonance imaging. An expensive magnetic resonance system is therefore required to check the nanoparticle concentration, said system also taking up a large amount of space.

The microcapsules or microspheres currently used generally consist of a magnetic core, namely the magnetic nanoparticle, on which chemotherapy drugs which cause the destruction of the tumor cells are deposited as a casing.

As mentioned above, these nanoparticles can be guided to or held at a particular target location, in other words in the tumor volume, by magnetic fields in the space, to act locally and selectively. To exercise such magnetic holding forces, non-homogeneous magnetic fields, particularly those with powerful gradients (gradient fields), are required, as can be produced for example by an electromagnet or even by permanent magnets.

The magnetic gradient field here is configured so that a spatial region known as the focal point exists, within which the holding forces produced are at a maximum. It is frequently problematic here to align this focal point with the target location, as the operation is frequently carried out manually at present based on image information or the like, which entails the risk that the treatment is not effective or is not sufficiently effective at the target location.

### SUMMARY OF THE INVENTION

The object of the invention is therefore to specify a microcapsule and a positioning method that permit better positioning and improved positioning verification.

To achieve this object according to the invention a microcapsule is provided for the local treatment of a tumor, comprising:

a support material forming a casing of the microcapsule,

an active agent that damages, in particular destroys, tumor cells,

a marker material suitable for use as an x-ray marker, and

at least one magnetic nanoparticle.

The invention therefore proposes a new structure for the microcapsules used, which can in particular be realized as microspheres having a spherical shape, and in addition also comprise a marker material suitable for use as an x-ray marker. The inventive microcapsule therefore not only allows the local application of an active agent which is concentrated with the aid of an external gradient field at the target location, i.e. in the tumor, it also ensures that the microcapsules are clearly visible in the x-ray or angiography image, so that it is possible at any time to check whether the microcapsules are disposed at the correct location. A plurality of such capsules is generally used for treatment purposes.

The active agent here can comprise a radioactive agent, in particular yttrium-90 and/or lutetium-177 and/or iron-59 and/or gallium-67, and/or an active chemotherapy agent, in particular doxorubicin and/or mitoxantrone. All these active agents permit very local, effective tumor treatment at the target location.

The support material used can be a biodegradable support material, in particular a polymer and/or a polyethylene glycol and/or a polyacrylate and/or a polyethylene oxide. The support material therefore ultimately forms a casing or support, in or on which the other materials are disposed. It is biodegradable and therefore leaves no harmful residues in the body of the patient.

It is also particularly advantageous for the marker material to be biodegradable, it being possible in particular to use iodine and/or barium sulfate. A microcapsule sub-material is therefore used which has low x-ray transparency and at the same time degrades biologically or can be excreted by the patient. Iodine is preferred here.

The nanoparticles can have an iron (III) oxide and/or iron (II,III) oxide base and/or be 10 to 300 nm, in particular 50 to 100 nm, preferably 80 nm, in size. They are then extremely suitable for being guided to the target location in an external gradient field or being held there.

As well as the microcapsule the object of the present invention is also achieved by a method for positioning a magnetic gradient field guiding magnetic nanoparticles, in particular magnetic nanoparticles of an inventive microcapsule, to a target location and/or holding them at the target location, in which process to position a focal point of the gradient field at the target location:

a catheter with at least one electromagnetic position sensor that in particular comprises at least one coil is guided to the target location, in particular a tumor, subject to image monitoring, and

the focal point is moved to the target location taking into account the signal from the position sensor.

It is therefore proposed that a catheter with an electromagnetic position sensor, for example one or a number of coils, should be used to decide based on the measured signal how the gradient field or its focal point should be positioned. To this end the catheter is first guided, subject to two-dimensional and/or three-dimensional image monitoring, to the target location, in particular into the tumor volume. The position of the position sensor therefore also corresponds to the position of the target location. The positioning of the gradient field relative to the target location is then changed by relative displacement of the target location with the tumor and the gradient field until the signal from the position sensor indicates that the focal point and the target location correspond. Finally the catheter coordinates are registered with the focal point of the magnetic gradient field. This allows the nanoparticles, and therefore also in particular the microcapsules, to be concentrated locally in an optimum manner at the target location, in particular in the tumor, and tumor cells to be destroyed with minimal side effects on surrounding healthy tissue.

Various known methods can be used here for image monitoring, for example the recording of fluoroscopy images in one plane (monoplanar system), the recording of fluoroscopy images in two planes (biplanar system) and also dynamic computed tomography methods or those having a high updating rate or methods related to computed tomography. Other facilities for image monitoring can of course also be used in addition to x-ray facilities.

Gradient fields are frequently used in which the gradients and therefore the holding forces ultimately produced are greatest in the region representing the focal point. If the position sensor, in particular the coil, is moved in the gradient field, an electromagnetic signal is induced, which is proportional to the gradient strength, so that in this instance the point of maximum gradient strength can easily be identified by means of the position sensor. In other words provision can be made for the focal point to be characterized by the maximum gradient and, once the catheter has been positioned, to be moved to the target location so that the signal measured at the position sensor is at a maximum. The catheter and therefore the target location, in particular the tumor, is then located at the focal point of the magnetic gradient field.

The focal point here can be displaced mechanically and/or electrically; however provision can also be made for the target location with the catheter disposed therein to be displaced mechanically.

In specific terms provision can be made when using electromagnets to produce the gradient field for the focal point to be displaced at least partially by corresponding actuation of the electromagnet(s). If therefore at least one electromagnet is provided as the magnetic means for producing the gradient field, said gradient field can be modified by way of the current applied thereto, it being possible in particular to displace the focal point in a particular direction or to a particular point. This in itself may be sufficient to locate the target location; whether this is possible in every instance mainly depends on the design and arrangement of the electromagnet(s). Additionally or alternatively provision can be made for the focal point to be displaced at least partially by mechanical displacement of the magnet producing the gradient field. This means that the magnet arrangement producing the gradient field is then moved mechanically relative to the target location and the catheter, for which purpose it is possible to use corresponding mechanical means, which should be similarly actuatable. Permanent magnets can of course also be used in this embodiment, to produce the gradient field.

Additionally or alternatively provision can also be made for the focal point to be displaced in the target object, in particular a patient, encompassing the target location at least partially by displacement of an apparatus supporting the target object, in particular a patient table. In this manner the target location with the catheter arranged there is therefore displaced actively to change the relative positioning. It is particularly advantageous for all the displacement methods mentioned here to be combined to achieve the greatest possible freedom and adaptability. Displacement of the apparatus supporting the target object can also be achieved by way of corresponding mechanical means.

In a particularly expedient embodiment of the present invention provision can be made for the in particular automatically performed displacement to take place in a directed manner taking account of a magnetic field map describing the gradient field and/or using an optimization method. It can already be known from the design and arrangement of the magnet(s) producing the gradient field, in particular in the form of a magnetic field map, what manner of form and in particular gradient profile the gradient field features, so that it can already be determined or at least qualified from the measurement values of the position sensor, where the catheter and therefore the target location are located relative to the focal point of the gradient field; such information, which can be calculated for example by a control facility, can advantageously be taken into account during displacement, in order to achieve the correspondence of focal point and target location in a quick and simple manner. Alternatively or additionally an optimization method can be employed, which ascertains for example based on short test displacements whether the desired change in the sensor signals is present so that an optimum displacement direction can be determined in order gradually to reach the target location.

In one particularly advantageous embodiment provision can also be made for a catheter also configured to inject microcapsules containing the magnetic nanoparticles to be used. The catheter then has to be moved to the target location anyway and serves there by means of the position sensor not only to position the gradient field but at the same time also to inject microcapsules containing the magnetic nanoparticles. In this instance the catheter is first guided to the target location subject to image monitoring. The gradient field is then suitably positioned so that its focal point corresponds to the target location, whereupon injection of the microcapsules, in particular the inventive microcapsules, provided to treat the tumor can take place. If the inventive microcapsules containing the marker material are used, it is then possible to check without any major difficulty, in particular by means of the x-ray facility used anyway to monitor the positioning of the catheter by means of images, whether the microcapsules are actually positioned at the correct point. This creates a general procedure, which permits optimum positioning and monitoring of positioning for the purposes of an optimum treatment result.

Finally the invention also relates to an apparatus for positioning a magnetic gradient field guiding magnetic nanoparticles, in particular magnetic nanoparticles of a microcapsule as claimed in one of claims 1 to 5, to a target location and/or holding them at the target location, comprising at least one magnet producing the gradient field, in particular an actuatable electromagnet, a catheter having at least one position sensor, in particular comprising at least one coil, and a control facility configured to perform the inventive method. The control facility is therefore configured to read out the signals from the position sensor, evaluate them in respect of the relative position of the focal point and the target location and actuate corresponding displacement means suitably, in order to position the gradient field in particular in a number of steps, so that its focal point corresponds to the target location. All the details relating to the inventive method can be applied in a similar manner to the inventive positioning apparatus.

Provision can therefore be made in particular for the inventive positioning apparatus also to comprise mechanical means that can be actuated by the control facility for the mechanical displacement of the magnet and/or at least one apparatus supporting a target object encompassing the target location as well as mechanical means that can be actuated by the control facility for the mechanical displacement of the apparatus. The control facility can then be configured for example to actuate the two mechanical means and the electromagnets so that the desired relative positioning of the focal point and target location results.

### BRIEF DESCRIPTION OF THE DRAWINGS

Further advantages and details of the present invention will emerge from the exemplary embodiments described in the following and with reference to the drawing, in which:

FIG. 1 shows an inventive microcapsule in a first embodiment,

FIG. 2 shows an inventive microcapsule in a second embodiment,

FIG. 3 shows a basic diagram of an inventive positioning apparatus, and

FIG. 4 shows a flow chart for a tumor treatment method.

### DETAILED DESCRIPTION OF THE INVENTION

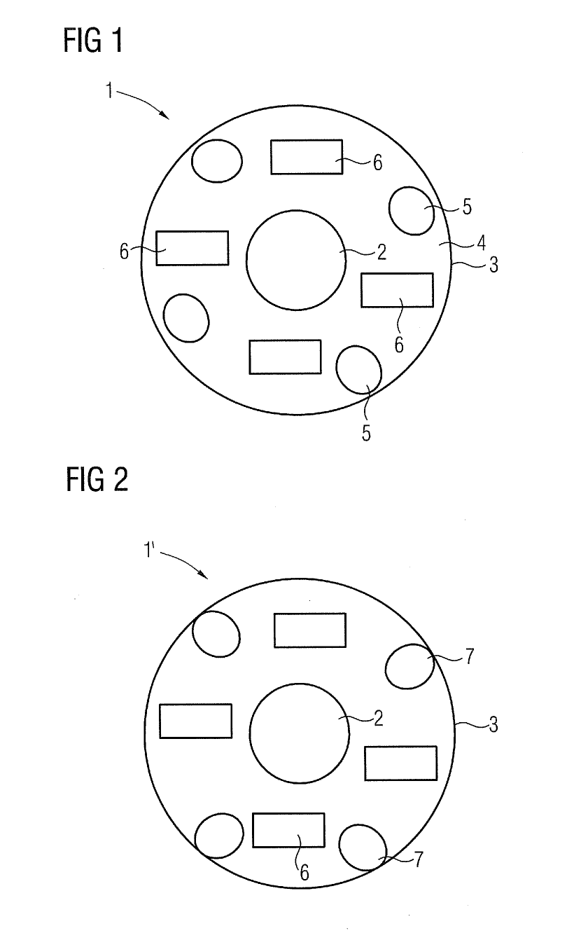

FIG. 1 shows a section through an inventive microcapsule 1 in a first embodiment. It comprises a magnetic nanoparticle 2 as the magnetic core, which can be formed on a basis of iron (III) oxide and/or iron (II,III) oxide. It has a diameter of around 80 nm. The outer casing 3 is formed by a support material 4. In the present example this is a biodegradable polymer.

Within the casing 3 the otherwise spherical microcapsule 1 also comprises a radioactive agent 5 (radioembolization agent), in this instance yttrium-90. Finally a marker material 6 is also provided within the casing 3, in this instance iodine, which serves as an x-ray marker.

The microcapsule 1 itself here has a diameter in the order of around five times the diameter of a red blood corpuscle, so that the microcapsule 1, when injected for example out of a catheter into a blood vessel supplying a tumor, remains lodged in the blood vessel within the tumor and can emit the radioactive radiation to destroy the tumor cells there. The nanoparticle 2 permits even more reliable positioning of microcapsules 1 in the tumor to be achieved, in that an external magnetic gradient field is used, the focal point, in other words the maximum gradient strength, of which corresponds to the tumor volume. The gradients here produce holding forces which prevent the microcapsule with the nanoparticles 2 leaving the target location, in other words the tumor volume. The gradient field can be positioned here by means of the inventive method described below.

The marker material 6 acting as an x-ray marker can finally be used to check the correct positioning of the microcapsules 1 by x-ray imaging, for example by means of fluoroscopy images or CT recordings. The marker material 6, which only has a very low level of transparency for x-ray radiation, allows the microcapsules 1 in the body of a patient to be identified clearly on the x-ray images.

FIG. 2 shows a further exemplary embodiment of an inventive microcapsule 1″, which only differs from the microcapsule 1 in that an active chemotherapy agent 7 is provided instead of the radioactive agent 5. This can be released within the tumor to bring about the destruction of tumor cells. In the present exemplary embodiment this is doxorubicin.

It should be pointed out here that embodiments of microcapsules are of course also conceivable in which both a radioembolization agent and an active chemotherapy agent are disposed.

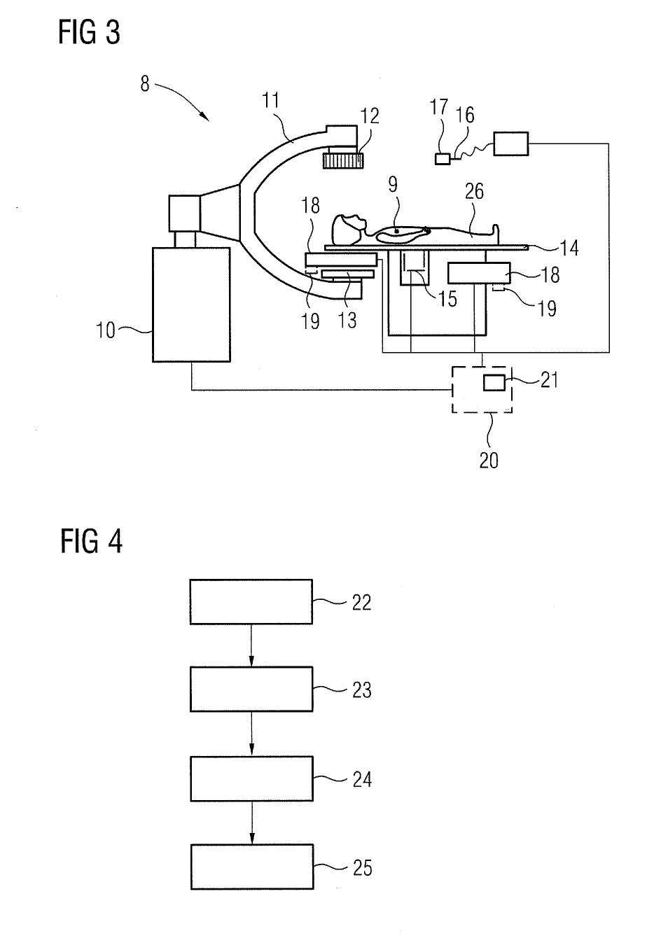

FIG. 3 shows a schematic diagram of an apparatus 8, in the present instance a complete treatment apparatus, which is however also configured in particular to position a magnetic gradient field so that the focal point of the gradient field corresponds to a target location 9, in this instance a tumor, within a patient 26. The gradient field here is embodied so that the focal point is characterized in that the maximum gradient strengths are present here, therefore the greatest holding forces are applied to nanoparticles 2.

The apparatus 8 comprises an x-ray facility 10, in this instance with a rotatable C-arms 11, on which an x-ray emitter 12 and an x-ray detector 13 are disposed opposite one another. The x-ray facility 10 can also be realized in the manner of a biplanar device with two C-arms 11. The C-arm 11 here can in particular be pivoted about the patient 26 disposed on a patient couch 14 in order to be able to record fluoroscopy images at different angles, from which it is then also possible to back calculate three-dimensional information, namely a three-dimensional image data set.

The abovementioned patient couch 14 has mechanical means 15, which permit displacement of the patient couch 14 in at least one spatial direction, in this instance even in three spatial directions. The apparatus 8 further comprises a catheter 16, which comprises on the one hand an electromagnetic position sensor 17 with three coils disposed perpendicular to one another, and on the other hand is also configured to inject microcapsules 1, 1′, as described above with reference to FIGS. 1 and 2, to the site of the catheter 16 in the patient 26.

Finally the apparatus 8 also comprises a magnet system for producing the magnetic gradient field, which in the present exemplary embodiment comprises two actuatable electromagnets 18, the position of which can also be changed by way of mechanical means 19. The focal point of the gradient field can therefore be displaced by corresponding actuation in the sense of applying a current to the electromagnets 18 but it is also possible to position the focal point in a different manner by mechanical displacement by means of the mechanical means 19.

A control facility 20 controls the operation of the entire apparatus 8.

Also stored in the control facility 20 in a corresponding storage apparatus is a magnetic field map 21, which describes the gradient field produced by the electromagnets 18 in different positions or at different currents.

The apparatus 8 can be used to perform the method described below, which also comprises a positioning method, which can be used to dispose the focal point of the gradient field automatically so that it corresponds to the target location 9, in other words the tumor volume. The control facility 20 is configured to perform this automatically.

In a first step 22, see FIG. 4, the catheter 16 is navigated to the target location 9 subject to image monitoring by the x-ray facility 10. Therefore at the end of step 22 the catheter 16 and the position sensor 17 are disposed at the target location 9.

In a step 23 the signals from the position sensor 17 are then evaluated automatically by the control facility 20 taking into account the magnetic field map 21, in order to change the relative position of gradient field and position sensor 17 and therefore target location 9 gradually by means of an optimization method so that the focal point of the gradient field corresponds to the position sensor 17 and therefore the target location 9. This position of the gradient field is then reached, as the focal point is characterized by the maximum gradient strength, when the signal from the position sensor 17 induced in the coils is at a maximum.

To change the relative position of the gradient field the control facility 20 is configured to actuate both the mechanical means 15, 19 and also the current applied to the electromagnets 18 accordingly.

If it is ascertained therefore at the end of step 23 that the focal point of the gradient field corresponds to the target location, in this instance in particular the tumor, in a step 24 the microcapsules 1 or 1′ are injected through the catheter 16 into the tumor. They are held in place there by the holding forces of the gradient field and can thus destroy tumor cells. In a step 25 it is checked, again using the x-ray facility 10, whether the microcapsules 1 or are actually present at the target location 9, this being advantageously possible due to the marker material 6. This permits simple, reliable and verifiable positioning of the gradient field and therefore of the injected microcapsules 1, 1′.

1.-13. (canceled)

14. A microcapsule for a local treatment of a tumor, comprising:
a casing comprising a support material;
an active agent that damages tumor cells;
a marker comprising a marker material that is suitable for use as an x-ray marker; and
a magnetic nanoparticle.

15. The microcapsule as claimed in claim 14, wherein the active agent is a radioactive agent that is selected from the group consisting of: lutetium-177, iron-59, gallium-67.

16. The microcapsule as claimed in claim 14, wherein the active agent is an active chemotherapy agent that comprises a doxorubicin and/or a mitoxantrone.

17. The microcapsule as claimed in claim 14, wherein the support material is a biodegradable support material that is selected from the group consisting of: a polymer, a polyethylene glycol, a polyacrylate, and a polyethylene oxide.

18. The microcapsule as claimed in claim 14, wherein the marker material is biodegradable that comprises an iodine and/or a barium sulfate.

19. The microcapsule as claimed in claim 14, wherein the magnetic nanoparticle is based on iron (III) oxide and/or iron (II,III) oxide with a diameter between 10 to 300 nm.

20. The microcapsule as claimed in claim 19, wherein the diameter of the magnetic nanoparticle is between 50 to 100 nm.

21. The microcapsule as claimed in claim 20, wherein the diameter of the magnetic nanoparticle is 80 nm.

22. A method for positioning a magnetic gradient field guiding a magnetic nanoparticle of a microcapsule to a target location, comprising:
guiding a catheter comprising an electromagnetic position sensor to the target location; and
displaying a focal point to the target location based on a signal from the position sensor.

23. The method as claimed in claim 22, wherein the focal point is characterized by a maximum gradient and is displaced to the target location after the catheter is guided to the target location so that a signal measured at the position sensor is at a maximum.

24. The method as claimed in claim 22, wherein the focal point is displaced at least partially by correspondingly actuating an electromagnet and/or at least partially by mechanically displaying a magnet producing the gradient field.

25. The method as claimed in claim 22, wherein the focal point is displaced at least partially by displaying an apparatus supporting a target object encompassing the target location.

26. The method as claimed in claim 22, wherein the focal point is displaced automatically based on a magnetic field map describing the gradient field and/or by an optimization method.

27. The method as claimed in claim 22, wherein the catheter injects the microcapsules containing the magnetic nanoparticle.

28. An apparatus for positioning a magnetic gradient field guiding a magnetic nanoparticle of a microcapsule to a target location, comprising:
a magnet that produces the magnetic gradient field;
a catheter comprising a position sensor; and
a control device that is configured to:
guide the catheter to the target location; and
display a focal point to the target location based on a signal from the position sensor.

29. The apparatus as claimed in claim 28, further comprising a mechanical device actuated by the control device for mechanically displaying the magnet.

30. The apparatus as claimed in claim 28, further comprising an apparatus supporting a target object encompassing the target location and a mechanical device actuated by the control device for mechanically displaying the apparatus.

31. The apparatus as claimed in claim 28, wherein the magnet comprises an actuatable electromagnet.

32. The apparatus as claimed in claim 28, wherein the position sensor comprises a coil.
